(19) United States
(12) Patent Application Publication
(10) Pub. No.: US 2013/0324992 A1
(43) Pub. Date: Dec. 5, 2013

(54) RADIO SCLEROTHERAPY SYSTEM

(71) Applicants: Ronald Bush, Dayton, OH (US); Brian J. Smith, Jacksonville Beach, FL (US); Anthony Johnson, Jacksonville, FL (US)

(72) Inventors: Ronald Bush, Dayton, OH (US); Brian J. Smith, Jacksonville Beach, FL (US); Anthony Johnson, Jacksonville, FL (US)

(73) Assignee: REFINE USA, LLC, Jacksonville Beach, FL (US)

(21) Appl. No.: 13/484,586

(22) Filed: May 31, 2012

(51) Int. Cl. A61B 18/18

(52) U.S. Cl. 606/33

(57) ABSTRACT

A radio frequency sclerotherapy system includes a needle assembly with a thin (26 to 32 gauge) solid conductive needle, about 1.25 inches long. A multilayer coating is applied over a proximal portion of the needle leaving an uncoated distal tip, about 0.10 inches long, exposed. The multilayer coating is an electric and thermal insulator. An electrical plug electrically coupled to the proximal end of the wire. The multilayer coating provides a palpable transition. A generator supplies voltages in the range of 300V to −100V, at frequencies of 1 MHz to 50 MHz, with voltage spikes having a time from rise to fall of up to 100 nS and a low voltage between voltage spikes for about twice the duration of the time from rise to fall of the voltage spikes.

### FIELD OF THE INVENTION

This invention relates generally to radio scelrotherapy, and, more particularly, to a radio sclerotherapy system that minimizes tissue damage and maximizes efficacy by providing a needle with a multilayer coating and exposed tip.

### BACKGROUND

The venous system of the leg consists of both a superficial and a deep component. The superficial component is located above the fascia, and the deep component is located below the fascia. The principal superficial veins are the lesser saphenous vein, which runs from the ankle to the knee, and the greater saphenous vein, which runs from the ankle to the groin. The superficial veins of the leg connect and empty into the deep veins via perforating veins, which pierce through the fascia separating the compartments of the leg.

Veins have one-way valves that occur every few inches along their course and are positioned to oppose back flow so deoxygenated blood can continue to flow in the direction of the heart. A varicose vein is a vein that has lost its elasticity. While any vein in the body may be affected, the superficial veins of the legs are by far the most frequently involved. These weakened veins dilate under the pressure of supporting a column of blood against the force of gravity. Varicose veins have a caliber greater than normal, and their valve cusps no longer meet. They are incompetent and result in reflux. Varicose veins impede proper circulation by permitting blood to flow away from the heart, decreasing the efficiency of the entire venous system, and leading to venous hypertension.

Sclerotherapy has been used for years to treat varicose veins. Injecting the unwanted veins with a sclerosing solution causes the target vein to shrink, and then dissolve over a period of weeks as the body naturally absorbs the treated vein. Potential complications include venous thromboembolism, visual disturbances, allergic reaction, thrombophlebitis, skin necrosis, and hyperpigmentation. For example, if a sclerosant is injected properly into the vein, there is no damage to the surrounding skin, but if it is injected outside the vein, tissue necrosis and scarring can result. Additionally, intense inflammatory reaction to the sclerotherapy agent in the area surrounding the injected vein can occur.

Radio frequency (RF) sclerotherapy, as an alternative to injecting sclerosing agents, uses high frequency current to heat an electrode inserted into the vein. The heated electrode coagulates vein constituents, causing the vein to shrink, and then dissolve over a period of weeks as the body naturally absorbs the treated vein. A needle electrode delivers radiofrequency energy to the vein wall causing it to heat. As the vein warms, it collapses and seals shut. While prior art RF sclerotherapy has been effective, the prior art suffers shortcomings relating to collateral damage and revival of the vein. Collateral damage includes tracking and marks where tissue other than the vein wall has been harmed by the heated electrode. Revival may be a result of inadequate heating of the targeted vein.

The invention is directed to overcoming one or more of the problems and solving one or more of the needs as set forth above.

### SUMMARY OF THE INVENTION

To solve one or more of the problems set forth above, in an exemplary implementation of the invention, a radio frequency sclerotherapy system includes a needle assembly with a thin solid conductive needle. The needle has a proximal portion and a distal tip and a diameter. The needle is electrically coupled to a distal end of a conductive wire. A multilayer coating is applied over the proximal portion of the needle. The distal tip is devoid of the multilayer coating. The multilayer coating includes an intermediate layer and a top layer. The intermediate layer is on the proximal portion of the needle and the top layer is on the intermediate layer. The multilayer coating is an electric and thermal insulator. An electrical plug electrically coupled to the proximal end of the wire.

The needle is 26 to 32 gauge, preferably about 29 gauge. The needle has a diameter of 0.01825 to 0.00925 inches, preferably about 0.011 inches. The length of the needle is from 0.75 to 2.0 inches, preferably about 1.25 inches. The length of the distal tip of the needle is from 0.05 to 0.25 inches, preferably about 0.10 inches, which is less than the diameter of many varicose veins.

The multilayer coating provides a palpable transition between the proximal portion and distal tip of the needle. The palpable transition is sensible when the distal tip is inserted into a vein up to the palpable transition.

The proximal portion of the needle is roughened with scratches on its surface for adherence of the intermediate layer.

The system may further include a generator electrically coupled to the wire. The generator controllably generates voltages in the range of 300V to −100V, at frequencies of 1 MHz to 50 MHz. The generator producing voltage spikes with a time from rise to fall of up to 100 nS. The generator maintains a low voltage between voltage spikes for about twice the duration of the time from rise to fall of the voltage spikes. The generator includes a wave synthesizer driving a power amplifier to produce the voltage spikes at the frequencies. The wave synthesizer comprises a master oscillator circuit with an astable multivibrator.

### BRIEF DESCRIPTION OF THE DRAWINGS

The foregoing and other aspects, objects, features and advantages of the invention will become better understood with reference to the following description, appended claims, and accompanying drawings, where:

FIG. 1 is a plan view of an exemplary sclerotherapy electrode needle assembly according to principles of the invention; and

FIG. 2 is a perspective view of an exemplary sclerotherapy electrode needle assembly according to principles of the invention; and

FIG. 3 is a plan view of an exemplary sclerotherapy electrode needle according to principles of the invention; and

FIG. 3A is a magnified view of an exemplary sclerotherapy electrode needle according to principles of the invention; and

FIG. 4 is a perspective view of an exemplary plug for a sclerotherapy electrode needle assembly according to principles of the invention; and

FIG. 5 is a schematic that conceptually illustrates an exemplary sclerotherapy electrode needle according to principles of the invention; and

FIG. 6 is a schematic that conceptually illustrates an exemplary sclerotherapy electrode needle inserted into a vein according to principles of the invention; and

FIG. 6A is a schematic that conceptually illustrates a magnified section of an exemplary sclerotherapy electrode needle according to principles of the invention; and

FIG. 7 is a graph that conceptually illustrates an exemplary waveform for electrically heating a sclerotherapy electrode needle according to principles of the invention; and

FIG. 8 is a schematic that conceptually illustrates components of an exemplary sclerotherapy electrode needle system according to principles of the invention; and

FIG. 9 is a schematic that conceptually illustrates electronic components of an exemplary sclerotherapy electrode needle system according to principles of the invention.

Those skilled in the art will appreciate that the figures are not intended to be drawn to any particular scale; nor are the figures intended to illustrate every embodiment of the invention. The invention is not limited to the exemplary embodiments depicted in the figures or the specific components, configurations, shapes, relative sizes, ornamental aspects or proportions as shown in the figures, except as expressly stated as a limit.

### DETAILED DESCRIPTION

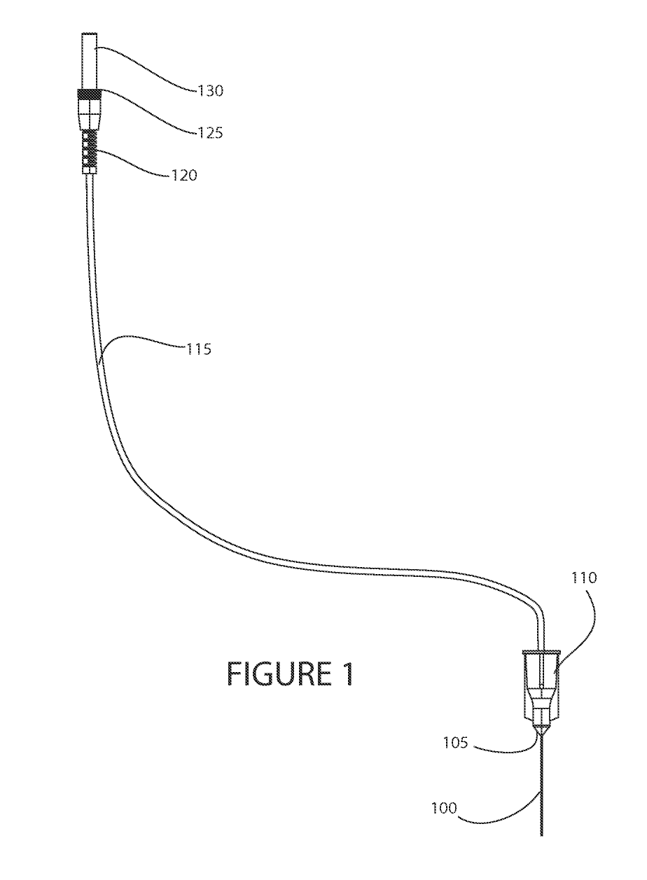

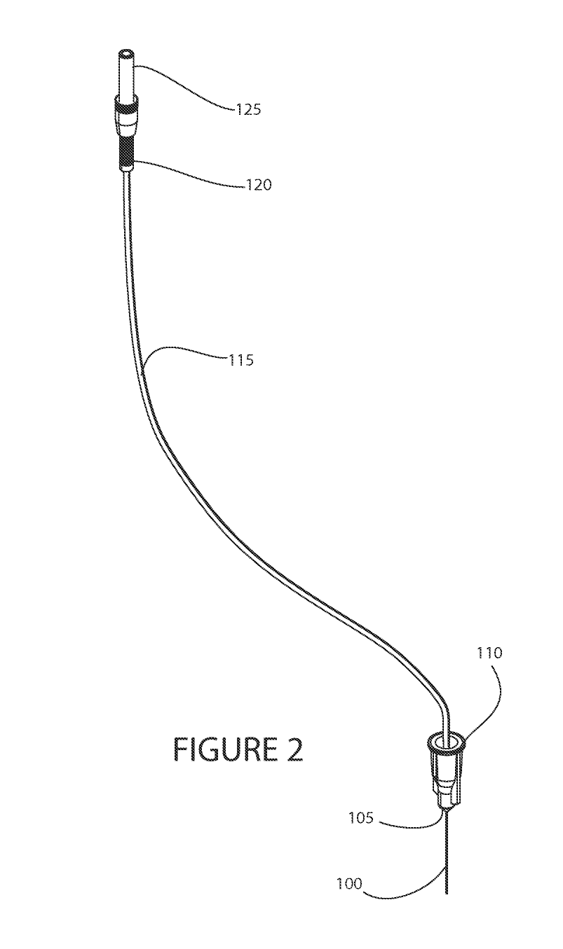

Referring to FIGS. 1 and 2, a plan view of an exemplary sclerotherapy electrode needle assembly according to principles of the invention. The assembly includes an electrically conductive needle 100, which is partially insulated as described below. A crimped conductive connector 105 joins the needle to an exposed conductive core of an insulated lead wire 115. A plastic handle 110 encases the crimped conductor and facilitates manipulation. A plug 130 with a collar 125 and a flexible neck 120 is provided to removably electronically couple the assembly to a controlled current source, as discussed below.

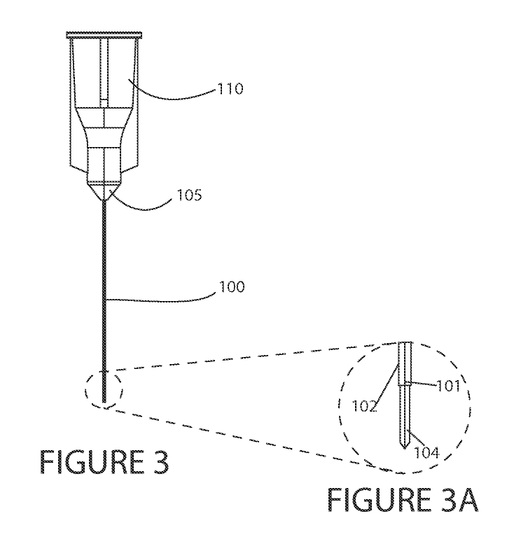

In FIGS. 3 and 3A close-up views of an exemplary needle and tip are provided. In an exemplary embodiment, the needle is comprised of SAE grade 304 stainless steel. The needle is solid with an outer diameter of about 26 to 32 gauge and a diameter of approximately 0.01825 to 0.00925 inches. In a particular preferred embodiment a 29 gauge needle or a needle having an outer diameter of about 0.011 inches is used. The length of the needle and the uninsulated tip of the needle is discussed below. As shown in the magnified view of FIG. 3A, the distal tip 104 of the needle 100 is uncovered while the proximal portion of the needle is coated with a thin insulator. The outer layer 101 of the insulator is shown in FIG. 3A. An inner layer is described below. As discussed below, the insulator is comprised of a plurality of layers of material applied in separate steps. The exposed distal tip 104 of the needle 100 is heated during use in RF sclerotherapy.

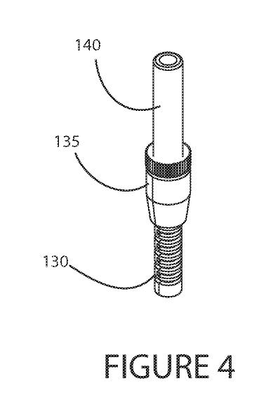

The plug 130 with a collar 125 and a flexible neck 120 is shown in FIG. 4. The plug 130 removably electronically couples the assembly to a controlled current source, as discussed below. The collar 135 limits the range of insertion into a mating coupling. An electrode within the hollow plug 130 establishes electrical contact. The flexible neck 120 allows bending without debonding or delamination between the wire and plug 130.

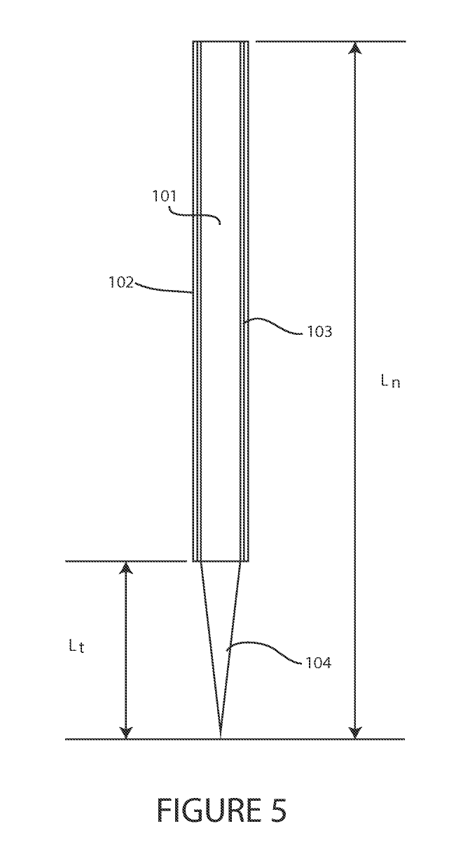

FIG. 5 illustrates the insulated proximal portion and exposed distal tip 104 of the needle 100. The insulated proximal portion includes a conductive core 101, an intermediate coating 103, and an outer coating 102. The intermediate coating 103 enhances bonding of the outer coating. The outer coating 102 provides insulation and reduced friction. The combined thickness of the outer 102 and intermediate coatings 103 is less than about one quarter of the diameter of the needle 100. Thus, the coatings do not impede insertion of the needle 100 into a patient. Nevertheless, the slight thickness of the multilayer coating 102, 103 is sensible as the tip 104 penetrates a vein wall. Thus, a practitioner may detect when the needle has been inserted sufficiently into a vein to be treated.

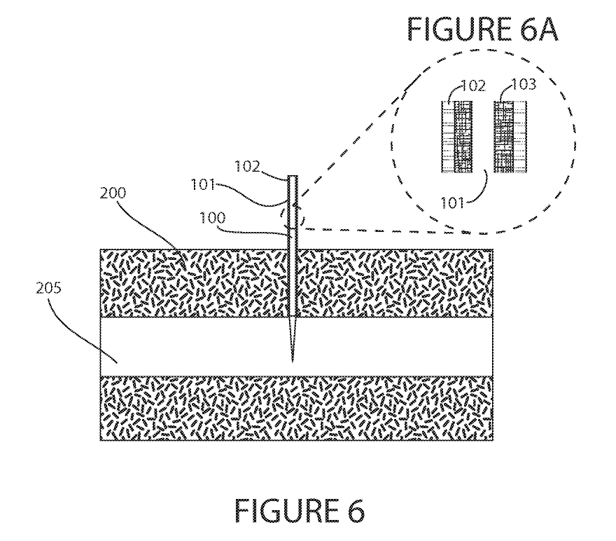

FIG. 6A also conceptually illustrates the insulated proximal portion including the conductive core 101, intermediate coating 103, and outer coating 102. In this embodiment, the surface of the needle to be coated is initially roughened to promote the adhering of the intermediate coating to the surface. The coated portion of the needle is roughened (e.g., sanded or sandblasted) to create microscratches on its surface. Then a primer coat, i.e., the intermediate coat, is applied. This coat is thin, enabling it to flow into the microscratches. The coated surface is then baked at high heat, causing the intermediate coat to solidify. Next the outer coat is applied over the intermediate coat. The outer coat will adhere to the intermediate coat, but not the uncoated tip. The needle is then baked, causing the outer coat to solidify. In a preferred embodiment the intermediate coat is a thin polytetrafluoroethylene primer and the outer coat is a polytetrafluoroethylene topcoat.

These materials offer good insulation properties along with excellent lubricity for easy insertion. The insulating effect of the multilayer coating is superior to that of an equivalent thickness single layer coating. Additionally, the bonding of the multilayer coating to the needle is superior to that of a single layer coating.

The insulation 53 must be thin because the needle 50 with the insulation 53 will be inserted into the vein to be treated. The thickness of the intermediate coat is 0.0005 to 0.001 inches, while the thickness of the outer coat is 0.0005 to 0.0015 inches. Thus the combined thickness is about 0.0010 to 0.0025 inches. As two layers are used and the bond between the layers is strong, the distal edge of the coatings maintains its integrity throughout insertion.

The overall length Lₙ of the needle 100 is from 0.75 to 2.0 inches, preferably 1.25 inches. The length Lₜ of the exposed (i.e., uncoated) tip of the needle is from 0.05 to 0.25 inches, preferably about 0.10 inches. For comparison, the diameter of a vein to be treated may vary roughly from about 1 mm (0.0394 inches) to about 15 mm (0.591 inches), depending upon condition and location. Most varicose veins are 3 mm or more in diameter. Thus, the exposed tip 104 of the needle is sized to penetrate the wall of a varicose vein without protruding entirely through the vein. As the tip 104 of the needle 100 emits the heat to cause coagulation, a needle with an exposed tip configured according to principles of the invention targets the vein with heat, while minimizing damage to surrounding tissue.

Referring now to FIG. 6, the needle is schematically shown extending through epidermis, dermis and subcutis 200 into a vein 205, through a vein wall, without penetrating the opposite side through the vein wall. The transition from the exposed tip to the insulated proximal portion of the needle is flush against the vein wall. The contact of the transition with the vein wall is palpable to a practitioner. The needle tip 104 delivers radiofrequency energy to the vein wall causing it to heat, collapse and seals shut.

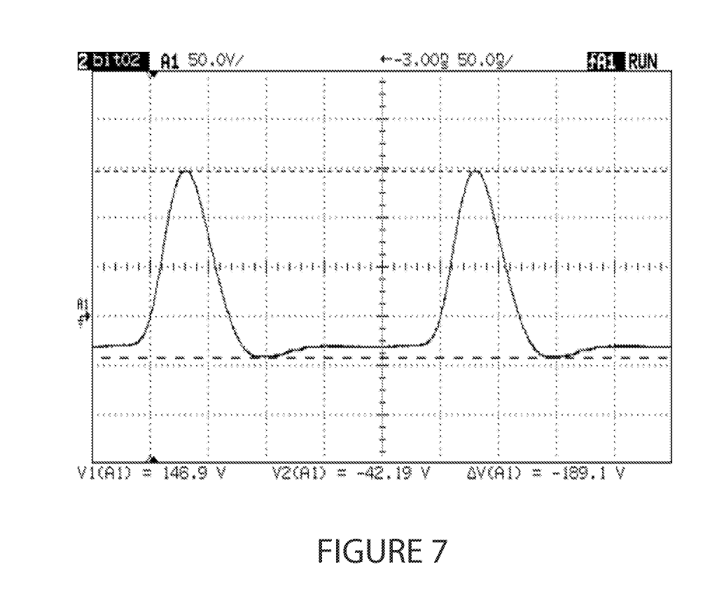

The electrosurgical unit is designed to create irreversible thermal alteration of tissues; that is, controlled thermal damage. The objective is to heat target vein walls to temperatures for times sufficient to yield the desired result. All of the physical effects of rf current are the result of elevated temperatures. The key observation is that the degree of alteration depends on both the temperature and the time of exposure. The particular radio frequency (RF) current is an important aspect of the invention. Although the needle assembly may work with a variety of current sources, in a particular preferred embodiment an electrosurgical generator produces voltage spikes with a rapid rise and fall at RF frequencies. The exemplary graph in FIG. 7 illustrates exemplary voltage spikes from a high of a maximum of about 146.9 V dropping to a low of about −42.19 V for a ΔV of about −189.1 V, with a plain between the successive spikes of about twice the duration from rise to fall for a spike. The rise and fall occurs within about 50 nS and is roughly symmetrical with a rise that is closely similar (i.e., about equivalent) in slope (i.e., ΔV/t) to the fall. The frequency is about 20 MHz. The rapid rise and fall, voltage differential and high frequency in conjunction with the needle structure described above, causes the needle to emit sufficient heat from the tip to treat veins, without unduly harming surrounding tissue.

Those skilled in the art will appreciate that the waveform in FIG. 7 is a nonlimiting example of an exemplary implementation. By way of example, maximum voltages in the range of 100V to 300V and minimum voltages of about 0V to −100V, and frequencies of about 1 MHz to 50 MHz, and times from rise to fall of about 3 to 100 nS, and plain times of about twice the rise to fall time, may be utilized in accordance with the principles of the invention. It is the short duration, symmetric high voltage spikes with a high frequency that drives the heats the needle in a manner effective for RF sclerotherapy.

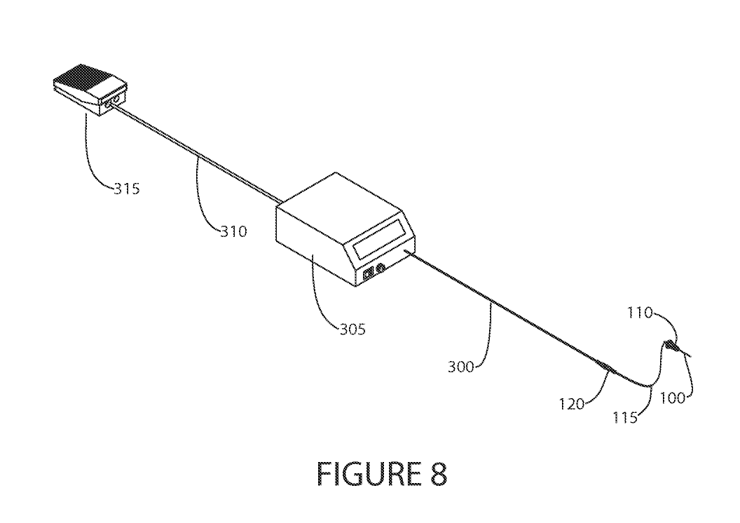

With reference to FIG. 8, an exemplary solid-state generator 305 is illustrated coupled to the needle assembly by a wire 300 terminating with a mating plug 120. A user control such as a foot pedal 315 is connected to the generator by wires 310. The generator may include on-off switches, power level adjustments and other user interface controls and a display.

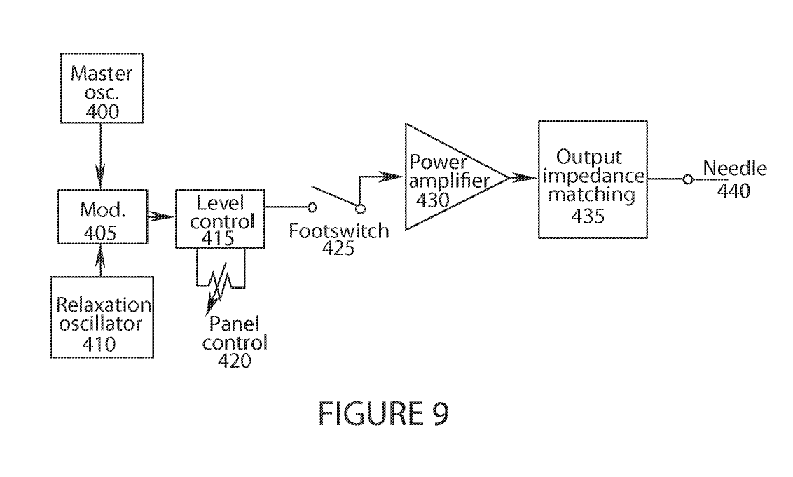

In a nonlimiting exemplary embodiment, a wave synthesis network drives a power amplifier output stage to produce the desired waveform. A block diagram of an exemplary solid-state electrosurgical generator is provided in FIG. 9. The fundamental frequency, is generated by a master oscillator circuit 400, such as an astable multivibrator. The primary oscillator acts as a clock or timing reference for the rest of the generator. An interrupted waveform is formed by gating the continuous oscillator output through a timing circuit comprising a modulator 405 and relaxation oscillator 410. The duty cycle of a waveform is the ratio of duration of the output burst to the time between initiation of bursts. A level control 415 coupled to a user interface power control 420 allows the user to set the power output. The modulated output is amplified using a power amplifier 430 when the activating switch (e.g., foot switch) is closed. The power amplifier may be comprised of bipolar junction transistors or HEXFET or VMOS transistors. Power output may be regulated by measuring the output voltage and current and adjusting the drive signal to compensate for changes in the equivalent load impedance, using output impedance matching 435. The regulated output is supplied to the needle for heating.

While an exemplary embodiment of the invention has been described, it should be apparent that modifications and variations thereto are possible, all of which fall within the true spirit and scope of the invention. With respect to the above description then, it is to be realized that the optimum relationships for the components and steps of the invention, including variations in order, form, content, function and manner of operation, are deemed readily apparent and obvious to one skilled in the art, and all equivalent relationships to those illustrated in the drawings and described in the specification are intended to be encompassed by the present invention. About or a similar qualifier signifies±10%. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

What is claimed is:

1. A radio frequency sclerotherapy system comprising:
a needle assembly comprising
a thin solid conductive needle, said needle having a proximal portion and a distal tip and a diameter,
an electrically conductive wire having a distal end and a proximal end, said wire being electrically coupled to the needle at a distal end of the wire,
a multilayer coating over the proximal portion of the needle, and
the distal tip being devoid of the multilayer coating,
the multilayer coating comprising an intermediate layer and a top layer, said intermediate layer being on the proximal portion of the needle and the top layer being on the intermediate layer, and said multilayer coating being an electric and thermal insulator.

2. A radio frequency sclerotherapy system according to claim 1, further comprising an electrical plug electrically coupled to the proximal end of the wire.

3. A radio frequency sclerotherapy system according to claim 1, said needle being 26 to 32 gauge.

4. A radio frequency sclerotherapy system according to claim 1, said needle being 29 gauge.

5. A radio frequency sclerotherapy system according to claim 1, said needle having a diameter of 0.01825 to 0.00925 inches.

6. A radio frequency sclerotherapy system according to claim 1, said needle having a diameter of about 0.011 inches.

7. A radio frequency sclerotherapy system according to claim 1, said needle having a diameter of 0.01825 to 0.00925 inches.

8. A radio frequency sclerotherapy system according to claim 1, the length of the needle being from 0.75 to 2.0 inches, and the length of the distal tip of the needle being from 0.05 to 0.25 inches.

9. A radio frequency sclerotherapy system according to claim 1, the length of the distal tip of the needle being less than the diameter of a varicose vein.

10. A radio frequency sclerotherapy system according to claim 1, said multilayer coating providing a palpable transition between the proximal portion and distal tip of the needle, said palpable transition being sensible when the distal tip is inserted into a vein up to the palpable transition.

11. A radio frequency sclerotherapy system according to claim 10, the length of the needle being about 1.25 inches, and the length of the distal tip of the needle being about 0.10 inches.

12. A radio frequency sclerotherapy system according to claim 11, the proximal portion of the needle being roughened with scratches on its surface.

13. A radio frequency sclerotherapy system according to claim 12, the intermediate layer being on the roughened proximal portion of the needle and the top layer being on the intermediate layer.

14. A radio frequency sclerotherapy system according to claim 13, said needle being 29 gauge.

15. A radio frequency sclerotherapy system according to claim 13, said needle having a diameter of about 0.011 inches.

16. A radio frequency sclerotherapy system according to claim 15, further comprising a generator electrically coupled to the wire, said generator controllably generating voltages in the range of 300V to −100V, at frequencies of 1MHz to 50 MHz.

17. A radio frequency sclerotherapy system according to claim 16, said generator producing voltage spikes with a time from rise to fall of up to 100 nS.

18. A radio frequency sclerotherapy system according to claim 17, said generator maintaining a low voltage between voltage spikes for about twice the duration of the time from rise to fall of the voltage spikes.

19. A radio frequency sclerotherapy system according to claim 18, said generator comprising a wave synthesizer driving a power amplifier to produce the voltage spikes at the frequencies.

20. A radio frequency sclerotherapy system according to claim 19, said wave synthesizer comprising a master oscillator circuit with an astable multivibrator.
